(19) United States
(12) Patent Application Publication
(10) Pub. No.: US 2024/0217920 A1
(43) Pub. Date: Jul. 4, 2024

(54) PROCESS FOR PRODUCING L-CARNITINE

(71) Applicants: HUBEI XUJIE PHARMACEUTICAL CO., LTD., Jingmen City (CN); CARNITECH LLC, North Brunswick, NJ (US)

(72) Inventors: Songzhou Hu, Princeton, NJ (US); Zhen Song, Shanghai (CN)

(21) Appl. No.: 18/090,005

(22) Filed: Dec. 28, 2022

(51) Int. Cl. C07C 227/02; C07C 227/42

(52) U.S. Cl. CPC C07C 227/02; C07C 227/42

(57) ABSTRACT

There is disclosed an improved process for the production of L-carnitine, comprising a step of decolorizing L-carnitinenitrile and removing residual alkali cyanide from a solution of L-carnitinenitrile with an oxidant, wherein the oxidant is preferably hydrogen peroxide or sodium hypochlorite.

### TECHNICAL FIELD

The present invention relates to an improved process for the production of L-carnitine, in particular, it relates to a process for decolorizing L-carnitinenitrile chloride and for removing residual cyanide from a solution of L-carnitinenitrile chloride.

### BACKGROUND OF THE INVENTION

L-carnitine is a naturally occurring quaternary ammonium acid involved in metabolism in most mammals, plants, and some bacteria. L-carnitine is also called vitamin BT and has a molecular structure of formula (I). It plays a critical role in energy production by transporting long-chain fatty acids into mitochondria so they can be oxidized to produce energy. L-carnitine also plays an important role in the regulation of metabolic pathways involved in skeletal muscle protein balance. Furthermore, L-carnitine acts as an anti-oxidant and as an anti-inflammatory compound. Therefore, L-carnitine finds wide applications as a nutritional supplement and feed additive.

There are many methods for the synthesis of L-carnitine, but only two processes are being used commercially.

The first process is the fermentative oxidation of gamma-butyrobetaine as depicted in the following reaction:

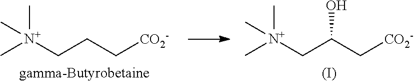

The second process starts from epichlorohydrin and accounts for the majority of L-carnitine produced. The reactions in the second process are described in the following scheme:

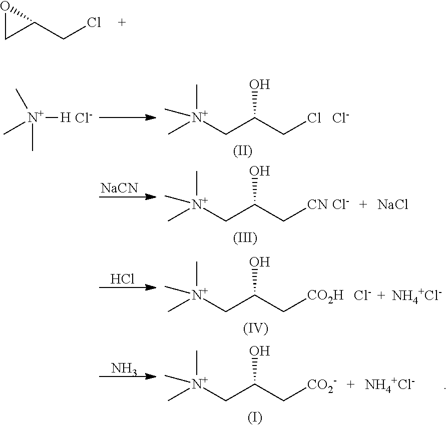

Racemic epichlorohydrin can be efficiently resolved into (S)-epichlorohydrin by using a Jacobsen Co(Salen) catalyst in high yield and high optical purity. (S)-epichlorohydrin is then reacted with trimethylamine hydrochloride to form L-3-chloro-2-hydroxypropyl trimethylammonium chloride of formula (II), which is subsequently reacted with sodium cyanide to form L-carnitinenitrile chloride of formula (III). These two steps can be carried out in a one-pot process without isolating the intermediate (II) in water. After isolation and purification, L-carnitinenitrile chloride is hydrolyzed in concentrated hydrochloric acid to yield L-carnitine and ammonium chloride. L-carnitine is finally isolated from this solution comprised of L-carnitine, excess hydrochloric acid, and ammonium chloride. The process according to this reaction scheme has been described in U.S. Pat. No. 9,096,493.

The reaction of epichlorohydrin with trimethylamine hydrochloride to form 3-chloro-2-hydroxyltrimethylammonium chloride is well known. In addition to the production of 3-chloro-2-hydroxyltrimethylammonium chloride, also known are several byproducts, such as 1,3-dichloro-2-propanol, 3-chloropropanediol, epoxy byproduct, and bis(trimethylammonium chloride)-2-propanol, i.e., the diquarternary salt.

U.S. Pat. No. 5,077,435 discloses a process to carry out the reaction of epichlorohydrin and trimethylamine hydrochloride in the presence of 1,3-dichloro-2-propanol as a cosolvent to reduce the formation of diquarternary salt, epoxy byproduct, and dichloropropanol. U.S. Pat. No. 5,463,127 further discloses that the reaction of epichlorohydrin and trimethylamine hydrochloride should be carried out at an initial pH of at least 8 by adding trimethylamine. U.S. Pat. No. 4,602,110 discloses a method to purify the product by crystallization from water-soluble alcohols.

U.S. Pat. No. 4,450,295 discloses a process of producing anhydrous trimethylamine chloride and then reacting it with epichlorohydrin to produce anhydrous 3-chloro-2-hydroxypropyl trimethylammonium chloride that is practically free of diquarternary bis(trimethylammonium chloride)-2-propanol.

U.S. Pat. No. 4,594,452 discloses a process for producing solid anhydrous 3-chloro-2-hydroxypropyl trimethylammonium chloride by carrying out the reaction of epichlorohydrin and trimethylamine hydrochloride in an organic solvent, which is a solvent for the two reactants, but a non-solvent for the product. Chloroform was disclosed to be a particularly suitable solvent.

JPH03287567 discloses in detail the reaction of chiral epichlorohydrin with trimethylamine hydrochloride to produce an optically active 3-chloro-2-hydroxylpropyl trimethylammonium chloride. When (S)-epichlorohydrin was reacted with an aqueous solution of trimethylamine hydrochloride, a solid product was obtained in a yield of 98.1% on the basis of trimethylamine. This crude product had a specific rotation of [α]_{D}²⁵=−23.4° (c=1.0, H₂O) and was purified by recrystallization twice in water to obtain a pure product of L-3-chloro-2-hydroxylpropyl trimethylammonium chloride, which had a specific rotation of [α]_{D}²⁵=−30.1° (c=1.0, H₂O) and a melting point of 215.5° C. When (R)-epichlorohydrin was used in the same reaction, a product was obtained in a yield of 96.8% with a specific rotation of [α]_{D}²⁵=+23.2° (c=1.0, H₂O). Recrystallization yielded a product of [α]_{D}²⁵=+29.5° (c=1.0, H₂O) and a melting point of 210.0-214.5° C. Despite the use of optically pure (S)- or (R)-epichlorohydrin in the reaction with trimethylamine hydrochloride, the product was not obtained as optically pure and the optical purity of the crude product was only about 78%. Hence, a partially racemized product was obtained even when optically pure (S)-epichlorohydrin was reacted with trimethylamine hydrochloride under the disclosed reaction conditions.

CN 102329243 discloses a continuous process in a tubular reactor for the reaction of (S)-epichlorohydrin dissolved in methanol with an aqueous solution of trimethylamine hydrochloride, wherein the ratio of the methanol solution of (S)-epichlorohydrin and the aqueous solution of trimethylamine hydrochloride is 1:0.92 (v/v). The product was obtained in a yield of 97% with a melting point of 190-191° C., which is much lower than 215° C. for a pure L-3-chloro-2-hydroxylpropyl trimethylammonium chloride. Hence, the method according to CN 102329243 did not yield an optically pure product.

In the reaction of L-3-chloro-2-hydroxylpropyl trimethylammonium chloride and alkali cyanide to produce L-carnitinenitrile chloride, alkali cyanide must be used in excess to fully convert L-3-chloro-2-hydroxylpropyl trimethylammonium chloride to L-carnitinenitrile chloride. As a result, there is a significant amount of alkali cyanide remained in a reaction solution of L-carnitinenitrile chloride. The concentration of residual alkali cyanide in a reaction solution can be as high as 1%. If not removed from a reaction solution, this large amount of residual alkali cyanide creates environmental problems and poses hazardous working conditions.

Moreover, the solution of L-carnitinenitrile chloride produced from a reaction of isolated L-3-chloro-2-hydroxylpropyl trimethylammonium chloride and alkali cyanide or from a one-pot reaction of trimethylamine hydrochloride, (S)-epichlorohydrin, and alkali cyanide, is invariably dark reddish and opaque. As a result, L-carnitinenitrile chloride isolated from this solution is colored and creates purification problem for downstream processing.

It is an objective of the present invention to ameliorate these disadvantages and to disclose an improved process for the production of L-carnitine by decolorizing L-carnitinenitrile chloride and by removing residual cyanide from a solution of L-carnitinenitrile chloride.

### SUMMARY OF THE INVENTION

The invention discloses an improved process for the production of L-carnitine by decolorizing L-carnitinenitrile chloride and by removing residual cyanide from a solution of L-carnitinenitrile chloride with an oxidant. Preferably, the oxidant is hydrogen peroxide or sodium hypochlorite.

### DETAILED DESCRIPTION OF THE INVENTION

The present invention relates to an improved process for the production of L-carnitine. In particular, it discloses a safe process for the production and isolation of L-carnitinenitrile chloride of improved quality.

The invention is accomplished by a surprising and unexpected discovery that an oxidant can be used to decolorize L-carnitinenitrile chloride and to remove residual cyanide from a solution of L-carnitinenitrile chloride.

There is no limit as to the source of L-carnitinenitrile chloride. L-carnitinenitrile chloride can be produced from a reaction of L-3-chloro-2-hydroxylpropyl trimethylammonium chloride of formula (II) and a source of cyanide. L-carnitinenitrile chloride can also be produced from a one-pot reaction of trimethylamine hydrochloride, (S)-epichlorohydrin, and a source of cyanide. L-carnitinenitrile chloride can be further produced by a reaction of L-2,3-epoxypropyl trimethylammonium chloride with a source of cyanide. In addition, a solution of L-carnitinenitrile chloride can be prepared by dissolving isolated L-carnitinenitrile chloride in a solution. Preferably, a solution of L-carnitinenitrile chloride is produced in an aqueous solution, optionally in the presence of an organic solvent.

Suitable sources of cyanide are selected from the group consisting of alkali cyanide, alkaline earth metal cyanide, zinc cyanide, and a cyanohydrin; wherein the alkali is lithium, sodium, or potassium, and wherein the alkaline earth metal is magnesium, calcium, or barium. Preferably, an alkali cyanide is used; more preferably, sodium cyanide is used. When an alkali cyanide is used, the product is an aqueous solution of L-carnitinenitrile and alkali chloride.

Suitable organic solvents are preferably water-soluble and are selected from the group consisting of methanol, ethanol, propanol, isopropanol, butanol, isobutanol, tert-butanol, methoxyethanol, ethoxyethanol, butoxyethanol, ethylene glycol, diethylene glycol, propylene glycol, tetrahydrofuran, dioxane, dimethyl formamide, dimethyl acetamide, dimethyl sulfoxide, N-methyl pyrrolidinone, 1,3-dimethylimidazolidinone, tetramethylurea, and a mixture thereof.

Suitable oxidants usable in the present invention are selected from the group consisting of hydrogen peroxide, urea hydrogen peroxide, performic acid, peracetic acid, perpropionic acid, perbenzoic acid, chloroperbenzoic acid, alkyl peroxide, dialkyl peroxide, benzoyl peroxide, chlorobenzoyl peroxide, alkali peroxide, alkaline earth metal peroxide, alkali percarbonate, alkali perborate, alkali hypochlorite, alkali hypobromite, alkaline earth metal hypochlorite, alkali chlorite, alkali chlorate, alkaline earth metal hypobromite, alkali persulfate, ammonium persulfate, N-chlorosuccinimide, N-bromosuccinimide, N-chlorophthalimide, N-bromophthalimide, 1,3-dichlorodimethylhydantoin, 1,3-dibromodimethylhydantoin, bromochlorodimethylhydantoin, trichloroisocyanuric acid, tribromoisocyanuric acid, alkali dichloroisocyanurate, alkali dibromoisocyanurate, alkali chloroisocyanurate, alkali bromoisocyanurate, and a mixture thereof, wherein the alkyl is a C₁-C₁₂ group; the alkali is lithium, sodium, potassium, or cesium; and the alkaline earth metal is magnesium, calcium, or barium.

Preferably, the oxidant is hydrogen peroxide or alkali hypochlorite. When hydrogen peroxide is used as the oxidant, water is the only byproduct left in an aqueous solution. Thus, no byproduct is introduced into the reaction solution. When alkali hypochlorite is used as the oxidant, alkali chloride is produced as a byproduct. However, this byproduct of alkali chloride is the same as the coproduct of L-carnitinenitrile chloride in a reaction of L-3-chloro-2-hydroxylpropyl trimethylammonium chloride and alkali cyanide. Thus, no different kind of byproduct is formed.

There is no limit as to the amount of an oxidant used in the process according to the present invention. It can be used in the range from 0.1% to 500% on the molar basis of L-carnitinenitrile chloride. Preferably, it is used in the range of 1% to 100%. More preferably, it is used in the range of 2% to 50%. Most preferably, it is used in the range from 2% to 20%.

The effective reaction temperature for an oxidant to function is from 20° C. to the boiling point of the solution. Preferably, the temperature is from 30° C. to 80° C. More preferably, the temperature is from 40° C. to 70° C. After the reaction of an oxidant in a solution of L-carnitinenitrile chloride is complete, excess oxidant can be decomposed by heating the solution to a higher temperature.

After a solution of L-carnitinenitrile chloride is treated with an oxidant in the process according to the present invention, a dark reddish and opaque solution of L-carnitinenitrile chloride becomes a clear and transparent solution. The color of the solution varies from light yellowish to colorless. In addition, the concentration of free cyanide in the solution of L-carnitinenitrile chloride falls to below 1 ppm. Preferably, the concentration of free cyanide is less than 0.5 ppm. More preferably, the concentration of free cyanide is less than 0.1 ppm. Most preferably, free cyanide is not detected.

L-carnitinenitrile chloride can be isolated from this treated aqueous solution by any method known to one skilled in the art. When L-carnitinenitrile chloride is isolated and separated from alkali chloride by using a lower alcohol, L-carnitinenitrile chloride is obtained as a nearly white crystalline product. A suitable lower alcohol is selected from the group consisting of methanol, ethanol, propanol, methoxyethanol, ethoxyethanol, isobutanol, tert-butanol, butanol, ethylene glycol, and a mixture thereof.

It has now been found that an aqueous solution of L-carnitinenitrile chloride after treatment with an oxidant in the process according to the present invention can be concentrated to crystallize L-carnitinenitrile chloride without using any organic solvent. L-carnitinenitrile chloride is obtained as a mixture with alkali chloride in a nearly white crystalline form and is free of impurities. This mixture of L-carnitinenitrile chloride and alkali chloride is particularly suitable for the production of L-carnitine by using a hydrolysis reaction.

It has further been found that an aqueous solution of L-carnitinenitrile chloride after treatment with an oxidant in the process according to the present invention can be used to produce L-carnitine without further purification. This embodiment is particularly advantageous, since the overall process for the production of L-carnitine becomes simplified and concise. Hence, L-carnitine can be produced in a cascade of reactions from L-3-chloro-2-hydroxylpropyl trimethylammonium chloride of formula (II) without isolating any intermediate.

L-carnitinenitrile chloride can be converted to L-carnitine by one of the known methods, for example, by using hydrochloric acid to hydrolyze the nitrile group in L-carnitinenitrile chloride. L-carnitine can be then isolated from the hydrolysis solution by one of the known methods by using, for example, ion exchange resin or electrodialysis.

After L-carnitine is isolated, the L-carnitine can be converted to L-carnitine L-tartrate by reacting with L-tartaric acid, L-carnitine fumarate with fumaric acid, and acetyl-L-carnitine hydrochloride with acetyl chloride, propionyl-L-carnitine hydrochloride with propionyl chloride, by processes known in the art.

The process according to the present invention can be carried out discontinuously, semi-continuously, or continuously.

### EXAMPLES

The following examples will illustrate the practice of this invention but are not intended to limit its scope.

### Comparative Example

To a round bottom flask were added 94 g of L-3-chloro-2-hydroxypropyl trimethylammonium chloride (0.5 mol) and 100 mL of water. After the solution was stirred and warmed to 35° C., 75 mL of aqueous solution containing 26.1 g of sodium cyanide was added in three portions over a period of about 1 hr. The temperature was maintained between 35° C. and 45ºC for 5 hours. The reaction solution changed from nearly colorless from the beginning to dark reddish and opaque. The solution was found to contain about 5,000 ppm of free cyanide.

To the reaction solution was added 5 g of activated carbon. After the suspension was stirred and heated to 80 ºC for about 1 hr., the suspension was filtered to remove activated carbon. The color of the filtration mother liquor solution remained unchanged to be dark reddish. The cyanide concentration was decreased to about 4,000 ppm.

### Example 1

To a round bottom flask were added 94 g of L-3-chloro-2-hydroxypropyl trimethylammonium chloride (0.5 mol) and 100 mL water. After the solution was stirred and warmed to 35° C., a solution containing 25.5 g of sodium cyanide was added in three portions over a period of about 1 hr. The temperature was maintained between 35° C. and 45ºC for 5 hours. The reaction solution changed from nearly colorless from the beginning to dark reddish and opaque. Afterwards, 4.5 mL of 35% hydrogen peroxide was added to the flask and the temperature was kept at 45ºC for about 1 hr. The dark reddish solution became a light yellowish and clear solution. A cyanide test showed 0.2 ppm of free cyanide.

### Example 2

To a round bottom flask were added 282 g of L-3-chloro-2-hydroxypropyl trimethylammonium chloride (1.5 mol) and 100 mL water to obtain a suspension. After the suspension was stirred and warmed to 35° C., 250 mL of a solution containing 75.6 g of sodium cyanide (1.54 mol) was added in three portions over a period of about 1 hr. The temperature was maintained between 35° C. and 45ºC for 5 hours. The reaction solution changed from nearly colorless from the beginning to dark reddish and opaque. Afterwards, 13 mL of 35% hydrogen peroxide was added to the flask and the temperature was kept at 45° ° C. for about 1 hr, then at 65° C. for 1 hr. The dark reddish solution became a light yellowish and clear solution. Free cyanide in the solution could not be detected (<0.1 ppm).

### Example 3

The reaction solutions of Examples 1 and 2 were combined and concentrated to a crystalline suspension. The suspension was then cooled to room temperature and filtered to obtain a crystalline mixture of L-carnitinenitrile chloride and sodium chloride. The mother liquor solution was repeatedly concentrated and filtered to obtain a crystalline mixture of L-carnitinenitrile chloride and sodium chloride. After drying, the solid weighted 452 g in a molar yield of 96%. The final mother liquor contained additional 24 g of solid material after evaporated to dryness.

### Example 4

To a round-bottom flask was added 300 mL of methanol and 100 g of the solid product obtained in Example 3. The suspension was heated to reflux and filtered hot to remove sodium chloride. The mother liquor solution was cooled to form a crystalline suspension. After filtration and drying, 65 g of L-carnitinenitrile chloride was obtained as a white crystalline solid. [α]_{D}²⁵=−25.9° (c=1.0, H₂O).

### Example 5

To a round bottom flask were added 50 mL of 30% hydrochloric acid and 47.2 g of a mixture of L-carnitinenitrile chloride and sodium chloride from Example 3. The solution was heated to 90ºC for about 4 hours and then cooled to room temperature. The suspension was then neutralized with aqueous solution of ammonia and applied to 1,000 mL of a strongly acidic resin bed. The absorbed L-carnitine was eluted with an aqueous solution of 3% ammonia. The L-carnitine fractions were combined and evaporated to dryness. The residue was dissolved in a minimal amount of anhydrous ethanol and L-carnitine was precipitated with acetone. After filtration, washing with acetone, and drying, the white crystalline product weighted 19.1 g. [α]_{D}²⁵=−30.4° (c=10, H₂O).

### Example 6

To a round bottom flask were added 188 g of L-3-chloro-2-hydroxypropyl trimethylammonium chloride (1.0 mol) and 200 mL water. After the solution was stirred and warmed to 35° C., a solution containing 51.6 g of sodium cyanide was added in three portions over a period of about 1 hr. The temperature was maintained between 35° C. and 45ºC for 5 hours. The reaction solution changed from nearly colorless from the beginning to dark reddish and opaque. Afterwards, the dark reddish reaction solution was diluted to 500 mL with water.

To each 50 mL of the dark reddish solution (0.1 mol) was added 0.005 mol (5%) of sodium hypochlorite, sodium percarbonate, sodium perborate, potassium persulfate (Oxone), ammonium persulfate, N-chlorosuccinimide, N-bromosuccinimide, 1,3-dichlorodimethylhydantoin, 1,3-dibromodimethylhydantoin, trichloroisocyanuric acid, respectively. In each case, after the solution or suspension was stirred at 50° C. for about 1 hr, the dark reddish became light yellowish to colorless and free cyanide could not be detected (<0.1 ppm).

It will be understood that the foregoing examples and explanation are for illustrative purposes only and that various modifications of the present invention will be self-evident to those skilled in the art. Such modifications are to be included within the spirit and purview of this application and the scope of the appended claims.

What is claimed is:

1. A process for the production of L-carnitine, comprising:
(a) adding an oxidant to a solution of L-carnitinenitrile chloride to decolorize and to remove residual cyanide;
(b) optionally isolating the L-carnitinenitrile chloride from the solution of step (a); and
(c) converting the L-carnitinenitrile chloride of step (a) or step (b) to L-carnitine.

2. The process according to claim 1, wherein the oxidant is selected from the group consisting of hydrogen peroxide, urea hydrogen peroxide, performic acid, peracetic acid, perpropionic acid, perbenzoic acid, chloroperbenzoic acid, alkyl peroxide, dialkyl peroxide, benzoyl peroxide, chlorobenzoyl peroxide, alkali peroxide, alkaline earth metal peroxide, alkali percarbonate, alkali perborate, alkali hypochlorite, alkali chlorite, alkali chlorate, alkali hypobromite, alkaline earth metal hypochlorite, alkaline earth metal hypobromite, alkali persulfate, ammonium persulfate, N-chlorosuccinimide, N-bromosuccinimide, N-chlorophthalimide, N-bromophthalimide, 1,3-dichlorodimethylhydantoin, 1,3-dibromodimethylhydantoin, bromochlorodimethylhydantoin, trichloroisocyanuric acid, tribromoisocyanuric acid, alkali dichloroisocyanurate, alkali dibromoisocyanurate, alkali chloroisocyanurate, alkali bromoisocyanurate, and a mixture thereof, wherein the alkyl is a C1-C12 group; the alkali is lithium, sodium, potassium, or cesium; and the alkaline earth metal is magnesium, calcium, or barium.

3. The process according to claim 1, wherein the oxidant is hydrogen peroxide.

4. The process according to claim 1, wherein the oxidant is sodium hypochlorite.

5. The process according to claim 1, wherein the solution of L-carnitinenitrile chloride is produced by a reaction of L-3-chloro-2-hydroxypropyl trimethylammonium chloride and alkali cyanide, wherein the alkali is lithium, sodium, potassium, and a mixture thereof.

6. The process according to claim 1, wherein the solution of L-carnitinenitrile chloride is produced by a reaction of trimethylamine hydrochloride, (S)-epichlorohydrin, and alkali cyanide, wherein the alkali is lithium, sodium, potassium, and a mixture thereof.

7. The process according to claim 1, wherein the solution of L-carnitinenitrile chloride is prepared by dissolving a solid L-carnitinenitrile chloride in a solution.

8. The process according to claim 1, wherein the solution of L-carnitinenitrile chloride is produced by a reaction of L-2,3-epoxypropyl trimethylammonium chloride and alkali cyanide, wherein the alkali is lithium, sodium, potassium, and a mixture thereof.

9. The process according to claim 1, wherein the oxidant is used in a molar amount of 0.1% to 500% of L-carnitinenitrile chloride.

10. The process according to claim 1, wherein the oxidant is used in a molar amount of 2% to 20% of L-carnitinenitrile chloride.

11. The process according to claim 1, wherein the reaction temperature of the oxidant in the solution of L-carnitinenitrile chloride is from room temperature to a boiling temperature of the solution of L-carnitinenitrile chloride.

12. The process according to claim 1, further comprising the production of L-carnitine L-tartrate by reacting the L-carnitine with L-tartaric acid.

13. The process according to claim 1, further comprising the production of L-carnitine fumarate by reacting the L-carnitine with fumaric acid.

14. The process according to claim 1, further comprising the production of acetyl-L-carnitine hydrochloride by reacting the L-carnitine with acetyl chloride.

15. The process according to claim 1, further comprising the production of propionyl-L-carnitine hydrochloride by reacting the L-carnitine with propionyl chloride.
